(19) United States
(12) Patent Application Publication
(10) Pub. No.: US 2011/0172506 A1
(43) Pub. Date: Jul. 14, 2011

(54) METHOD AND APPARATUS FOR PORTABLY MEASURING CALORIE CONSUMPTION USING IDC SENSOR

(75) Inventor: SANG-HUN LEE, YONGIN-SI (KR)

(73) Assignee: SAMSUNG ELECTRONICS CO., LTD., SUWON-SI (KR)

(21) Appl. No.: 13/004,429

(22) Filed: Jan. 11, 2011

(30) Foreign Application Priority Data
Jan. 12, 2010 (KR) 10-2010-0002833

(51) Int. Cl. A61B 5/00

(52) U.S. Cl. 600/307

(57) ABSTRACT

Disclosed is a method for portably measuring calories, the method including determining if an electric field is formed through an Inter-Digital Capacitor (IDC) sensor in contact with skin; extracting an amount of sweat generated from the skin using the formed electric field; and measuring the calories using the extracted resultant value.

### PRIORITY

This application claims priority under 35 U.S.C. §119(a) to an application entitled “Method and Apparatus for Portably Measuring Calorie Using IDC Sensor” filed in the Korean Industrial Property Office on Jan. 12, 2010, and assigned Serial No. 10-2010-0002833, the contents of which are incorporated herein by reference.

### BACKGROUND OF THE INVENTION

1. Field of the Invention

The present invention relates generally to a multi-sensor calorie measurer for measuring calories consumed during a workout or other actions using various sensors, and more particularly, to an apparatus and a method for precisely measuring an amount of consumed calories using an Inter-Digital Capacitor (IDC) sensor.

2. Description of the Related Art

As interest in the fields of health, fitness, and dieting has recently increased, they maintain their health through exercise such as running, walking, or the like. While performing a workout, they want to know how many calories are consumed. In order to satisfy such a requirement, and to more scientifically manage a workout and body fat, portable calorie measuring apparatuses have been produced, which can measure the amount of calories consumed during daily life or a workout by being simply attached to the body.

Initially, such portable calorie measuring apparatuses were mainly passometer-type apparatuses based on an acceleration sensor. However, recently, portable calorie measuring apparatuses have been developed to employ various kinds of sensors and algorithms, which provide relatively exact results in various situations and various types of workouts in addition to running and walking by comprehensively extracting and analyzing bio-signals.

One type of sensor included in a portable calorie measuring apparatus, which is for extracting bio-signals from a body to measure calories, includes a heart rate measuring sensor for measuring the number of times a heart beats in a preset time, an acceleration sensor for measuring the extent of motions generated by running or walking, a heat flux sensor for measuring the extent of body heat generated by a workout, and a sweating sensor for measuring the extent of sweat generated by a workout. Hereinafter, a process for measuring calories using the heart rate measuring sensor from among the sensors will be described.

FIG. 1 is a flow chart illustrating a conventional calorie measuring process using a heart rate.

Referring to FIG. 1, in step 101, a user inputs his information such as sex, age, weight, or the like into a portable calorie measuring apparatus including a heart rate measuring sensor. In step 103, the portable calorie measuring apparatus, which has received the information, measures a heart rate before a workout using the heart rate measuring sensor, thereby measuring calories consumed by the user before the workout in real time, and measuring the amount of calories for a predetermined time. In step 105, when the user starts to perform a workout, the portable calorie measuring apparatus continuously measures the heart rate of the user using the heart rate measuring sensor, measures calories in real time during the user's workout, and measures the amount of calories consumed during the time of workout. In step 107, when the workout is finished, the apparatus measures and displays the amount of calories consumed by the workout.

A portable calorie measuring apparatus with only one sensor, from among various sensors including the above described heart rate measuring sensor, has an advantage in that its size can be minimized and its shape is simple, but has a limitation in that it is difficult to exactly measure calories on various types of exercises and motions. For example, a portable calorie measuring apparatus using only an acceleration sensor can be relatively exact in measuring calories in some exercises, which have a correlation between the extent of motion and an exercise amount, such as running or walking, but shows a low accuracy in anaerobic exercises using a dumbbell, a barbell, etc. performed in a limited space, due to a low extent of motion compared to an actual exercise amount.

A multi-sensor portable calorie measuring apparatus including two or more sensors has an advantage in that its accuracy in calorie measurement is higher than a portable calorie measuring apparatus having only one sensor because corresponding sensors calculate resultant values in various conditions in accordance with predetermined algorithms. For example, in a case where a user rides on an exercise bicycle, since an acceleration component of the sensor worn around an arm is hardly changed, it is impossible to exactly measure calories using only an acceleration sensor. Meanwhile, since the body generates heat and sweat, the values of a heat flux sensor and a sweating sensor can be used to more accurately calculate consumed calories. However, due to the inclusion of two or more sensors, all of the resultant values of the sensors have to be reflected in calorie measurement. This may cause a problem in that cost of development is increased and thus a unit cost is increased.

Also, the apparatus may cause an inaccurate result as a portable apparatus due to the difficulty in minimization of its size, and the complication of a product structure. Especially, in a case of a Galvanic Skin Response (GSR) sensor used as a sensor for measuring the amount of sweat in a commercialized product, although it is very useful in exactly calculating the amount of consumed calories by recognizing the state of a user's motion, it has a problem in that its occupied area or its size is much larger than other sensors, thereby significantly limiting the minimization of a product. This is because the sensor employs a method of measuring an impedance by flowing current through skin, and thus has to include two metallic electrodes apart from each other. Then, the area of the electrodes has to be larger than a predetermined size because a contact area between the electrodes and the skin is required to be large in order to obtain a high-quality signal.

Accordingly, in order to develop a portable calorie measuring apparatus capable of satisfying portability and performing exact measurement of calories in various workout conditions, a new-type sensor is required which has performance characteristics better than a conventional sensor, and at the same time can minimize a product due to significantly reduced sizes of its components.

### SUMMARY OF THE INVENTION

Accordingly, in order to measure an amount of sweat generated, an important function of a high-performance multi-sensor calorimeter, the present invention provides an apparatus and a method for measuring calories, in which the apparatus has performance characteristics, better than a conventional GSR sensor employing a skin conduction method, and having a very small size. Thus, the apparatus can satisfy portability and at the same time can exactly measure calories in various workout conditions.

In accordance with an aspect of the present invention, there is provided a method for portably measuring calories, the method including determining if an electric field is formed through an Inter-Digital Capacitor (IDC) sensor in contact with skin; extracting an amount of sweat generated from the skin using the formed electric field; and measuring the calories using the extracted resultant value.

In accordance with another aspect of the present invention, there is provided an apparatus for portably measuring calories, the apparatus including an IDC sensor in contact with skin, which is for measuring the calories based on an amount of sweat generated from the skin; a control part for controlling the IDC sensor to perform or stop measurement, and to transmit a measured resultant value; a display part for displaying the measured resultant value received from the control part, in real time; and a memory part for storing the measured resultant value received from the control part.

### BRIEF DESCRIPTION OF THE DRAWINGS

The above and other aspects, features and advantages of the present invention will be more apparent from the following detailed description taken in conjunction with the accompanying drawings, in which:

FIG. 1 is a flow chart illustrating a conventional calorie measuring process using a heart rate;

FIG. 2 is a block diagram illustrating the inside of a multi-sensor calorie measuring apparatus including an IDC sensor according to an embodiment of the present invention;

FIG. 3 illustrates the structure of an IDC sensor according to an embodiment of the present invention;

FIG. 4 illustrates the formation of an electric field in an IDC sensor according to an embodiment of the present invention;

FIG. 5 is a flow chart illustrating a process of measuring an amount of sweat generated using an IDC sensor according to an embodiment of the present invention; and

FIG. 6 is a graph illustrating a change in measurement values of an IDC sensor related to a measured amount of sweat according to an embodiment of the present invention.

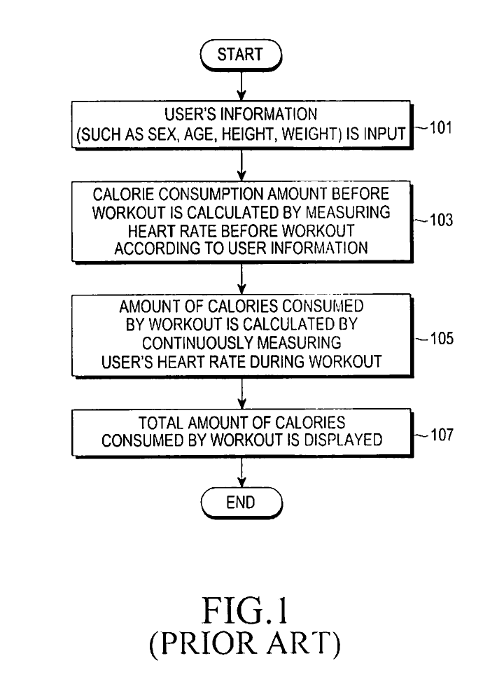

### DETAILED DESCRIPTION OF EMBODIMENTS OF THE PRESENT INVENTION

Hereinafter, preferred embodiments of the present invention will be described in detail with reference to the accompanying drawings. Particulars found in the following description of the present invention, such as specific configuration elements, etc., are provided only to help comprehensive understanding of the present invention, and it is obvious to those skilled in the art that various changes in form and details may be made in the particulars without departing from the spirit and scope of the invention.

As mentioned above, in the prior art, a portable calorie measuring apparatus including one sensor has a limitation in that it is difficult to exactly measure calories under certain measurement conditions. Also, from among multi-sensor portable calorie measuring apparatuses including two or more sensors, an apparatus including a Galvanic Skin Response (GSR) sensor for measuring the amount of sweat GSR has a problem in that it may cause an inaccurate result as a portable apparatus because there is a limitation in minimizing a product due to the size of the sensor itself, and the structure is complicated.

Accordingly, in order to solve the problems mentioned in the prior art, the present invention provides a method for utilizing an Inter-Digital Capacitor (IDC) sensor for measuring the amount of sweat generated in a multi-sensor calorie measurer, the sensor being designed to exactly measure calories in various workout conditions and at the same time to allow a product to be minimized.

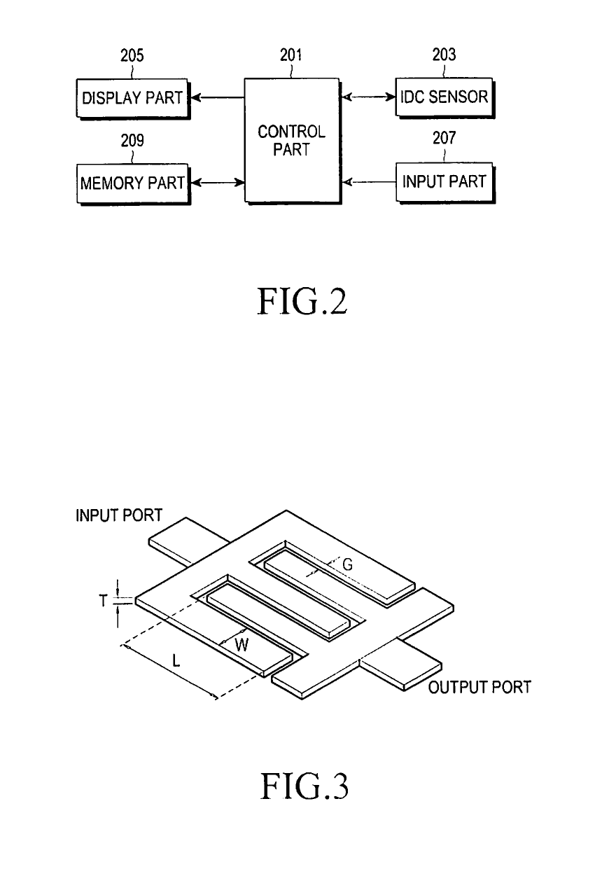

FIG. 2 is a block diagram illustrating the inside of a multi-sensor calorie measuring apparatus including an IDC sensor according to an embodiment of the present invention. The multi-sensor calorie measuring apparatus shown in FIG. 2 includes a control part 201, an IDC sensor 203, a display part 205, an input part 207, and a memory part 209.

Referring to FIG. 2, the input part 207 performs a role of receiving the input of one or more commands of calorie measurement, such as start, stop, and finish, to the multi-sensor calorie measuring apparatus by a user. The commands can be input by preset buttons. When the multi-sensor calorie measuring apparatus includes a touch-screen, the commands can be input using the touch-screen.

The display part 205 performs a role of displaying a result of calorie measurement according to a preset method. Also, when one or more commands are input from the input part 207, the display part 205 performs a role of displaying the commands. The result of calorie measurement may be displayed in real time, or may be displayed by calculating the total amount of calories consumed for a predetermined time.

The control part 201, when receiving one or more commands of calorie measurement, such as start, stop, and finish, from the input part 207, performs a role of transmitting such one or more commands to the display part 205, and controlling the IDC sensor 203 according to the one or more commands. For example, when receiving a command for starting calorie measurement, the control part 201 orders the IDC sensor 203 to measure the amount of generated sweat. Then, when receiving a sweat generation amount measured by the IDC sensor 203, the control part 201 measures an amount of calories, and transmits the measured result to the display part 205. Also, the control part 201 performs a role of transmitting the received sweat generation amount, and the measured calories, to the memory part 209.

The memory part 209 performs a role of storing the sweat generation amount, and the measured calories, received from the control part 201.

Although not shown in FIG. 2, the multi-sensor calorie measuring apparatus of the present invention may further include, besides the IDC sensor 203, an acceleration sensor, a heart rate measuring sensor, a heat sensor, or the like, and can exactly measure calories in various conditions by compositely using the sensors together with the IDC sensor 203.

FIG. 3 illustrates the structure of an IDC sensor according to an embodiment of the present invention. As shown in FIG. 3, the IDC sensor may include two electrodes, in which the two electrodes come into contact with skin while a voltage is applied, and an electric field is generated, thereby forming a capacitor structure. The measured capacitance varies according to a characteristic (such as a dielectric constant) of a through-path of the electric field. The formation range of the electric field varies according to the values of G indicating an interval between the respective electrodes, W indicating a width thickness of gold foil of the electrodes, and T indicating a length thickness of gold foil of the electrodes. For example, when W as a width thickness of electrode gold foil, and T as a length thickness are in the relation of “T<<W”, the penetration depth of the electric field is represented by a relational expression of (G+W)/π=0.637 W. Accordingly, when W is appropriately selected, it is possible to design the sensor in such a manner that the electric field can pass through the epidermal portion of skin. Sweat is generated and secreted from a horny layer corresponding to the epidermal portion of skin. Thus, when the sensor is designed in such a manner that the electric field can pass through the epidermal portion of skin, it is possible to exactly measure the amount of sweat generated.

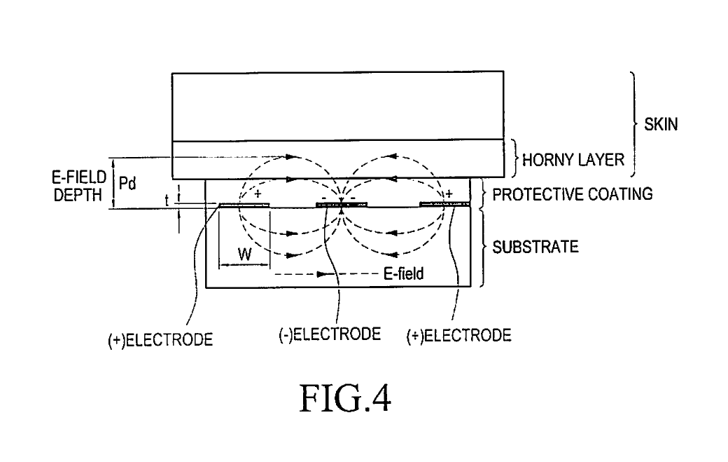

FIG. 4 illustrates the formation of an electric field in an IDC sensor according to an embodiment of the present invention. FIG. 4 shows a lateral cross-sectional view of the IDC sensor shown in FIG. 3. The IDC sensor includes two electrodes, a substrate, and protective coating. The drawing shows a process of forming an electric field when the IDC sensor comes into contact with a user's skin.

Referring to FIG. 4, in the IDC sensor, in order to inhibit a short which may be caused by the two electrodes' contact with skin, a protective coating is mounted on the two electrodes. The protective coating has no effect on the formation of the electric field or the electric field's sensing of the user's sweat as a material of the protective coating, glass or paralyne is used.

The depth of the electric field means the penetration depth of the electric field, illustrated in FIG. 3. In general, since the thickness of the epidermal portion of a human is relatively uniform, it is possible to be relatively exact in measuring the amount of sweat generated by only setting the initial thicknesses of W and T. In general, in order to exactly measure the amount of sweat generated, T has to be significantly thinner than W.

When a dielectric substance is generated, the two electrodes form an electric field as shown in FIG. 4, from (+) electrode to (−) electrode. As the amount of sweat generated from skin increases, the dielectric constant increases. Thus, the value of the capacitor is increased. Accordingly, the extent of sweat generation may be calculated by extracting the value from the capacitor.

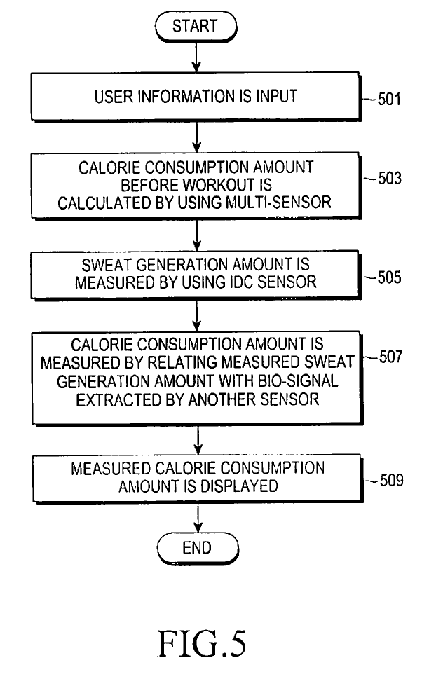

FIG. 5 is a flow chart illustrating a process of measuring an amount of sweat generated using an IDC sensor according to an embodiment of the present invention.

Referring to FIG. 5, in step 501, a user inputs his information such as sex, age, weight, and the like, into the multi-sensor calorie measuring apparatus including the IDC sensor. In step 503, the multi-sensor calorie measuring apparatus, which has received the information, measures the extent of sweat generated from the user's skin before a workout, at least one bio-signal using the IDC sensor, and at least one other sensor mounted within the measuring apparatus. Based on this, the multi-sensor calorie measuring apparatus measures calories consumed by the user before the workout in real time and then measures the amount of calories for a predetermined time. For example, in a case of a multi-sensor calorie measuring apparatus including the IDC sensor, and a heart rate measuring sensor, the calories consumed by the user are measured by compositely applying a measured amount of sweat and the number of heartbeats.

In step 505, when a workout is started, the multi-sensor calorie measuring apparatus continuously measures the amount of sweat generated from the user's skin using the IDC sensor, during the user's workout, and stores the resultant value measured during the workout in real time. At this time, bio-signals measured by another sensor are also measured and stored in real time. In step 507, by relating the measured extent of sweat to the bio-signals measured by another sensor, the amount of calories consumed during a workout time is measured. In step 509, when the workout is finished, the amount of calories consumed by the workout is displayed.

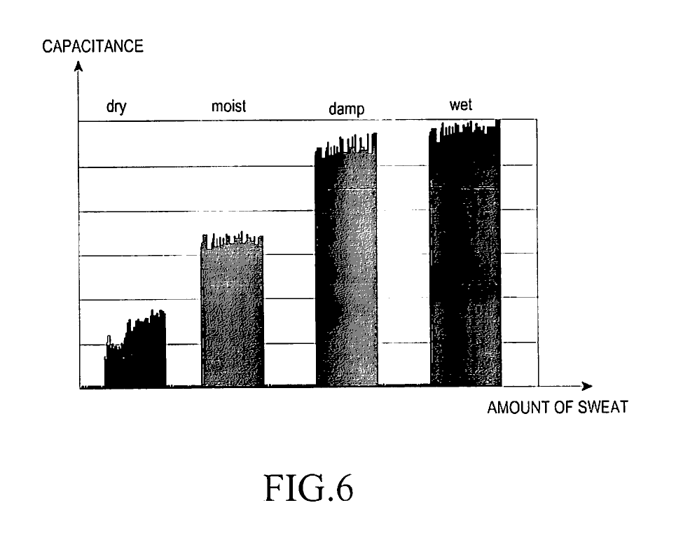

FIG. 6 is a graph illustrating a change in measurement values of an IDC sensor related to a measured amount of sweat according to an embodiment of the present invention.

Referring to FIG. 6, it can be found that a graph height is relatively low when little sweat exists on the skin, while a graph height increases as the amount of sweat on the skin increases. This indicates that the capacitance, that is, a value measured by the IDC sensor, is gradually increased according to the amount of sweat generated. Such information may be used to measure the amount of sweat generated. Also, by relating the information with bio-signals extracted from another sensor, it is possible to more accurately measure the amount of calories consumed.

In the present invention, a multi-sensor calorie measurer employs an IDC sensor in order to measure the generation of sweat during a workout. The measurer has an advantage in that portability is increased due to a small size of a sensor portion performing a role of measuring the amount of sweat generated, a additional sensor capable of improving the accuracy of measurement of consumed calories can be easily mounted due to increased free space, and the comfort of wearing the device is improved due to a reduced contact area between a sweat measuring sensor and the skin.

While the invention has been shown and described with reference to certain embodiments thereof, it will be understood by those skilled in the art that various changes in form and details may be made therein without departing from the spirit and scope of the invention as defined by the appended claims. Accordingly, the scope of the invention is defined by the appended claims and equivalents thereto, not by the described embodiments.

What is claimed is:

1. A method for portably measuring calories, the method comprising the steps of:
(a) determining if an electric field is formed through an Inter-Digital Capacitor (IDC) sensor in contact with skin;
(b) extracting an amount of sweat generated from the skin using the formed electric field; and
(c) measuring the calories using the extracted resultant value.

2. The method as claimed in claim 1, further comprising receiving input of a user's at least one information item in order to measure the calories.

3. The method as claimed in claim 1, wherein step (a) comprises applying a voltage between two electrodes included in the IDC sensor in contact with the skin; and determining if the electric field is formed in a preset range.

4. The method as claimed in claim 3, wherein a formation range of the electric field varies according to a thickness of the two electrodes included in the IDC sensor.

5. The method as claimed in claim 3, wherein in applying the voltage, the sweat generated from the skin functions as a dielectric substance to apply the voltage.

6. The method as claimed in claim 1, wherein step (c) comprises:
sequentially extracting the amount of the sweat generated from the skin within a preset time; and
measuring the calories by calculating a capacitance by the extracted amount of the sweat generated, and referring to information on a change in the capacitance.

7. The method as claimed in claim 1, wherein in step (c), the calories are measured by relating the amount of the sweat generated, measured using the IDC sensor, with bio-signals measured by another sensor.

8. An apparatus for portably measuring calories, the apparatus comprising:
an Inter-Digital Capacitor (IDC) sensor in contact with skin, which is for measuring an amount of sweat generated from the skin;
a control part for controlling the IDC sensor to perform or stop measurement, and to transmit a measured resultant value;
a display part for displaying the measured resultant value received from the control part, in real time; and
a memory part for storing the measured resultant value received from the control part.

9. The apparatus as claimed in claim 8, further comprising an input part for receiving a user's at least one information item in order to measure the calories.

10. The apparatus as claimed in claim 8, wherein the IDC sensor forms an electric field using two electrodes included in the IDC sensor in contact with the skin, and measures the calories based on the amount of the sweat generated from the skin.

11. The apparatus as claimed in claim 10, wherein a voltage is applied between the two electrodes in contact with the skin, and the electric field is formed in a preset range.

12. The apparatus as claimed in claim 11, wherein a formation range of the electric field varies according to a thickness of the two electrodes.

13. The apparatus as claimed in claim 11, wherein the voltage is applied using the sweat generated from the skin as a dielectric substance.

14. The apparatus as claimed in claim 8, wherein the IDC sensor comprises a protective coating mounted on two electrodes in order to inhibit a short caused by the two electrodes' contact with the skin.

15. The apparatus as claimed in claim 8, wherein the IDC sensor sequentially measures the amount of the sweat generated from the skin within a preset time, calculates a capacitance using the measured amount of the sweat, and measures the calories by referring to a change in the capacitance.

16. The apparatus as claimed in claim 8, which measures the calories by relating the amount of the sweat generated, measured using the IDC sensor, with bio-signals measured by another sensor.
